(19) United States
(12) Patent Application Publication
(10) Pub. No.: US 2007/0231247 A1
(43) Pub. Date: Oct. 4, 2007

(54) Method for Diluting Hypochlorite

(76) Inventors: Steven E. Bromberg, Livermore, CA (US); Elias A. Shaheen, Danville, CA (US); William L. Smith, Pleasanton, CA (US); Kenneth Vieira, Livermore, CA (US)

Correspondence Address: THE CLOROX COMPANY, P.O. BOX 24305, OAKLAND, CA, 94623-1305, US

(21) Appl. No.: 11/762,254

(22) Filed: Jun. 13, 2007

Related U.S. Application Data
(63) Continuation-in-part of application No. 10/828,571, filed on Apr. 20, 2004, now abandoned.
Application No. 10/828,571 is a continuation-in-part of application No. 10/806,522, filed on Mar. 23, 2004.

(51) Int. Cl. C01B 11/04

(52) U.S. Cl. 423/473

(57) ABSTRACT

This invention relates to methods of diluting hypochlorite and hypochlorous acid compositions with purified water to produce stable compositions. These compositions can be used to treat allergen containing surfaces, hard surfaces, food contact surfaces, hospital surfaces, food surfaces, kitchen surfaces, bathroom surfaces, human surfaces, animal surfaces, military equipment, transportation equipment, children's items, plant surfaces, seeds, outdoor surfaces, soft surfaces, air, wounds, and medical instruments.

### CROSS REFERENCE TO RELATED APPLICATIONS

The present application is a continuation-in-part of Co-pending application Ser. No. 10/828,571, which was filed Apr. 20, 2004, entitled “Method for Diluting Hypochlorite”, which is a continuation-in-part of Co-pending application Ser. No. 10/806,522, which was filed Mar. 23, 2004, entitled “Methods for deactivating allergens and preventing disease”, and both incorporated herein.

### BACKGROUND OF THE INVENTION

1. Field of the Invention

This invention relates to methods for diluting hypohalous acid, hypohalous acid salt, and compositions containing these actives. The resulting compositions are useful for disinfecting (for example, water, environmental hard and soft surfaces, human and animal surfaces), sanitizing, sterilizing medical devices, controlling odor, deactivating allergens, and controlling mold. The resulting compositions can be applied by a variety of means, including vaporizing, spraying, soaking, and applying by means of an impregnated substrate. The resulting compositions can be applied on hard surfaces, soft surfaces and in the air.

2. Description of the Related Art

The compositions of the invention are generally non-hazardous, non-irritating and non-sensitizing to the skin, non-irritating to the eyes, not harmful if swallowed and show no evidence of mutagenic activity. U.S. Pat. Appl. 2002/0182262 to Selkon states that high concentration of hypochlorite ions are no longer recommended for use in the treatment of leg ulcers due to their irritant and painful effects and impairment of cell growth which outweigh their therapeutic value, resulting in these preparations falling out of use. Attempts have been made to reduce the alkaline effect of the high hypochlorite ion content of these solutions, e.g. by the use of suitable buffers, but have been found to be ineffective in such circumstances. U.S. Pat. No. 6,936,220 to Hoshino et al. found that reduction in concentration of the chlorine compound in an attempt to ensure safety and prevent damage to objects involves difficulties in obtaining a formulation with satisfactory storage stability. That is, the activity would be reduced considerably due to the surrounding temperature, light (ultraviolet light), a third component adhered to a container, etc., a pigment present in a container material, and so on, and chlorine gas generation with decomposition of the chlorine compound. Thus, it has been difficult with a disinfecting deodorant comprising an aqueous solution of the chlorine compound to achieve sufficient disinfecting and deodorizing effects in such a low concentration range as to satisfy requirements for safety and the like. U.S. Pat. No. 6,936,220 to Hoshino et al. further finds that the composition is preferably from pH 9.5 to 11. If the pH is below 8, the disinfecting deodorant has a fear of generating chlorine gas with decomposition of the chlorine-containing oxidizing agent and fails to have sufficient storage stability. U.S. Pat. No. 5,281,280 to Lisowski et al. finds that concentrations below 2.75% are ineffective against mold, mildew and algae.

Potential uses for the inventive compositions and methods include for dishwashing, for example U.S. Pat. Appl. 2003/0216271 to Scheper et al.; for hospital environments and medical instruments, for example U.S. Pat. No. 6,632,347 to Buckley et al. and U.S. Pat. No. 6,126,810 to Fricker et al.; for wound healing, for example U.S. Pat. Appl. 2003/0185704 to Bernard et al. and U.S. Pat. No. 6,426,066 to Najafi et al.; for disinfecting or sterilizing objects such as medical instruments, for example U.S. Pat. No. 6,623,695 to Malchesky et al.; for disinfecting and deodorizing the air, for example U.S. Pat. Appl. 2002/0179884 to Hoshino et al.; for water purification, for example U.S. Pat. No. 6,296,744 to Djeiranishvili et al.; for removal of mold and mildew, for example U.S. Pat. No. 5,281,280 to Lisowski et al. Each of these references is hereby incorporated within in its entirety. Based on the prior art examples, various novel formulations and methods have been discovered for hypohalous acid, hypohalous acid salt, and compositions containing these actives.

### SUMMARY OF THE INVENTION

In accordance with the above objects and those that will be mentioned and will become apparent below, one aspect of the present invention is a method for producing a stable dilute composition, said composition selected from the group consisting of hypohalous acid, hypohalous acid salt, and combinations thereof, said method comprising the steps of providing a source of active material from the group consisting of hypohalous acid, hypohalous acid salt, hypohalous acid generating species, hypohalous acid salt generating species, and combinations thereof; and diluting said source with purified water and inorganic buffer to give said stable dilute composition having a pH between 4 and less than 8; wherein said stable dilute composition has an available chlorine concentration of between 40 ppm to about 400 ppm; wherein said stable dilute composition does not contain additives selected from the group consisting of surfactants, alcohols, amino compounds, thio compounds, hydroxyacids, olefinic compounds, fragrances or combinations thereof.

In accordance with the above objects and those that will be mentioned and will become apparent below, another aspect of the present invention comprises a method for producing a stable dilute composition, said composition selected from the group consisting of hypohalous acid, hypohalous acid salt, and combinations thereof, said method comprising the steps of providing a source of active material from the group consisting of hypohalous acid, hypohalous acid salt, hypohalous acid generating species, hypohalous acid salt generating species, and combinations thereof; and diluting said source with purified water to give said stable dilute composition; wherein said stable dilute composition has an available chlorine concentration of between 40 ppm to about 1200 ppm; wherein said stable dilute composition does not contain additives selected from the group consisting of surfactants, alcohols, hydroxyacids, fragrances or combinations thereof.

In accordance with the above objects and those that will be mentioned and will become apparent below, another aspect of the present invention comprises a method for producing a stable dilute composition, said composition selected from the group consisting of hypohalous acid, hypohalous acid salt, and combinations thereof, said method comprising the steps of preparing a first solution having an active halogen content of greater than about 0.5% as available chlorine; and diluting said first solution with purified water to give a second solution; wherein said second solution has an available chlorine concentration of between 40 ppm to about 400 ppm; wherein said second solution retains at least 50% of the available chlorine concentration at a storage temperature of 120° F. over 27 days; wherein said stable dilute composition does not contain additives selected from the group consisting of surfactants, alcohols, hydroxyacids, fragrances or combinations thereof.

Further features and advantages of the present invention will become apparent to those of ordinary skill in the art in view of the detailed description of preferred embodiments below.

### DETAILED DESCRIPTION

Before describing the present invention in detail, it is to be understood that this invention is not limited to particularly exemplified systems or process parameters that may, of course, vary. It is also to be understood that the terminology used herein is for the purpose of describing particular embodiments of the invention only, and is not intended to limit the scope of the invention in any manner.

All publications, patents and patent applications cited herein, whether supra or infra, are hereby incorporated by reference in their entirety to the same extent as if each individual publication, patent or patent application was specifically and individually indicated to be incorporated by reference.

It must be noted that, as used in this specification and the appended claims, the singular forms “a,” “an” and “the” include plural referents unless the content clearly dictates otherwise. Thus, for example, reference to a “surfactant” includes two or more such surfactants.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

In the application, effective amounts are generally those amounts listed as the ranges or levels of ingredients in the descriptions, which follow hereto. Unless otherwise stated, amounts listed in percentage (“%'s”) are in weight percent (based on 100% active) of the cleaning composition alone, not accounting for the substrate weight. Each of the noted cleaner composition components and substrates is discussed in detail below.

As used herein, the term “substrate” is intended to include any web, which is used to clean an article or a surface. Examples of cleaning sheets include, but are not limited to, mitts, webs of material containing a single sheet of material which is used to clean a surface by hand or a sheet of material which can be attached to a cleaning implement, such as a floor mop, handle, or a hand held cleaning tool, such as a toilet cleaning device.

As used herein, “wiping” refers to any shearing action that the substrate undergoes while in contact with a target surface. This includes hand or body motion, substrate-implement motion over a surface, or any perturbation of the substrate via energy sources such as ultrasound, mechanical vibration, electromagnetism, and so forth.

The term “cleaning composition”, as used herein, is meant to mean and include a cleaning formulation having at least one surfactant.

The term “surfactant”, as used herein, is meant to mean and include a substance or compound that reduces surface tension when dissolved in water or water solutions. The term “surfactant” thus includes anionic, nonionic, cationic and/or amphoteric agents.

Hypohalous Acid and Salts

Suitable hypohalous acids and salts may be provided by a variety of sources, including compositions that lead to the formation of positive halide ions and/or hypohalite ions; hypohalous acid, hypohalous acid salt, hypohalous acid generating species, hypohalous acid salt generating species; as well as compositions that are organic based sources of halides, such as chloroisocyanurates, haloamines, haloimines, haloimides and haloamides, or mixtures thereof. These compositions may also produce hypohalous acid or hypohalite species in situ. Suitable hypohalous acids and salts for use herein include the alkali metal and alkaline earth metal hypochlorites, hypobromites, hypoiodites, chlorinated trisodium phosphate dodecahydrates, potassium and sodium dichloroisocyanurates, potassium and sodium trichlorocyanurates, N-chloroimides, N-chloroamides, N-chlorosulfamide, N-chloroamines, chlorohydantoins such as dichlorodimethyl hydantoin and chlorobromo dimethylhydantoin, bromo-compounds corresponding to the chloro-compounds above, and compositions which generate the corresponding hypohalous acids, or mixtures thereof.

In one embodiment wherein the compositions herein are liquid, said hypohalite composition comprises an alkali metal and/or alkaline earth metal hypochlorite, or mixtures thereof. Compositions may comprise an alkali metal and/or alkaline earth metal hypochlorite selected from the group consisting of sodium hypochlorite, potassium hypochlorite, magnesium hypochlorite, lithium hypochlorite and calcium hypochlorite, and mixtures thereof.

The hypohalous acids and salt composition may be an equilibrium mixture of hypochlorous acid and sodium hypochlorite. The active species is present in an amount from above zero to about 15 weight percent of the composition, or from about 0.001 weight percent (10 ppm) to about 10 weight percent of the composition, or from about 0.005 (50 ppm) to about 5 weight percent of the composition. The compositions may have between 40 ppm to about 600 ppm available chlorine, or between 40 ppm to about 500 ppm available chlorine, or between 40 ppm to about 400 ppm available chlorine, or between 40 ppm and 1200 ppm, or from 40 ppm to less than 200 ppm, or from 40 ppm to less than 100 ppm, or between 50 ppm to about 400 ppm available chlorine, or between 50 ppm and 1200 ppm, or from 50 ppm to less than 200 ppm, or from 50 ppm to less than 100 ppm, or between 100 ppm to about 600 ppm available chlorine, or between 100 ppm to about 500 ppm available chlorine, or between 100 ppm to about 400 ppm available chlorine, or between 400 ppm to about 500 ppm available chlorine.

The amount of available halogen oxidant in the composition is determined by placing samples of the composition into about 50 milliliters of distilled water, followed by addition of about 10 milliliters of a 10 weight/weight percent solution of potassium iodide and addition of about 10 milliliters of a 10 volume percent solution of sulfuric acid, the resulting mixture being well stirred. The resulting yellow to brown solution, whose color is the result of oxidation of free iodide ion (I⁻) to molecular iodine (I₂), was then volumetrically titrated to an essentially colorless endpoint by addition of standardized 0.1 Molar sodium thiosulfate (Na₂S₂O₃) titrant. Calculation then expresses the result as percent of available molecular chlorine (Cl₂), that is to say assigning two equivalents per mole of titrated hypohalite oxidant. Stability results are then expressed by repeated assays over time using identically prepared samples resulting from the same composition, normalized to 100 percent representative of the starting available chlorine measured initially.

During the course of evaluating various oxidants and antimicrobials for their allergen deactivating ability, we have found that a very dilute solution (on the order of 40-80 ppm, or 40-200 ppm) containing a substantial amount of hypochlorous acid can effectively deactivate allergens. Presumably the low levels of oxidant are still able to break up the allergen proteins, rendering them biologically inert.

While still extremely biocidally effective, the low concentration and nearly neutral pH (6.9) hypochlorous acid virtually eliminates surface damage. There is no sticky residue that can affect the feel of fabrics and there may be minimal dye damage. The solution may be aerosolized to treat air directly, or applied to surfaces.

Aerosols are known to have a low collision rate between denaturant and allergen particles. As a result, the denaturant must be used in high concentrations to be effective. Using this approach with conventional allergen denaturants, which may be irritating or fragranced at high levels, can cause health problems.

Nonwoven Substrate

In one embodiment, the composition is applied to a substrate composed of nonwoven fibers or paper. The term nonwoven is to be defined according to the commonly known definition provided by the “Nonwoven Fabrics Handbook” published by the Association of the Nonwoven Fabric Industry.

Methods of making nonwovens are well known in the art. Generally, these nonwovens can be made by air-laying, water-laying, meltblowing, coforming, spunbonding, or carding processes in which the fibers or filaments are first cut to desired lengths from long strands, passed into a water or air stream, and then deposited onto a screen through which the fiber-laden air or water is passed. The air-laying process is described in U.S. Pat. App. 2003/0036741 to Abba et al. and U.S. Pat. App. 2003/0118825 to Melius et al. The resulting layer, regardless of its method of production or composition, is then subjected to at least one of several types of bonding operations to anchor the individual fibers together to form a self-sustaining substrate. In the present invention the nonwoven substrate can be prepared by a variety of processes including, but not limited to, air-entanglement, hydroentanglement, thermal bonding, and combinations of these processes.

In one aspect, dry substrates can be provided with dry or substantially dry cleaning or disinfecting agents coated on or in the multicomponent, multilobal fiber layer. In addition, the substrates can be provided in a pre-moistened and/or saturated condition. The wet substrates can be maintained over time in a sealable container such as, for example, within a bucket with an attachable lid, sealable plastic pouches or bags, canisters, jars, tubs and so forth. Desirably the wet, stacked substrates are maintained in a resealable container. The use of a resealable container is particularly desirable when using volatile liquid compositions since substantial amounts of liquid can evaporate while using the first substrates thereby leaving the remaining substrates with little or no liquid. Exemplary resealable containers and dispensers include, but are not limited to, those described in U.S. Pat. No. 4,171,047 to Doyle et al., U.S. Pat. No. 4,353,480 to McFadyen, U.S. Pat. No. 4,778,048 to Kaspar et al., U.S. Pat. No. 4,741,944 to Jackson et al., U.S. Pat. No. 5,595,786 to McBride et al.; the entire contents of each of the aforesaid references are incorporated herein by reference. The substrates can be incorporated or oriented in the container as desired and/or folded as desired in order to improve ease of use or removal as is known in the art. The substrates of the present invention can be provided in a kit form, wherein a plurality of cleaning substrates and a cleaning tool are provided in a single package. Suitable systems are described in Co-pending application Ser. No. 10/632,573 which was filed Aug. 1, 2003, entitled “Disinfecting article with extended efficacy”, and incorporated herein.

Surfactants

The composition of the invention may contain surfactants, but even hypochlorite stable surfactants are generally not stable in dilute hypochlorite compositions, such as those containing 40 to 200 ppm. In general, surfactants such as amine oxide, alkylpolyglycoside, aryl sulfonates, quaternary ammonium compounds, and betaines are not compatible with dilute hypochlorite compositions for long term stability, especially dilute hypochlorite compositions of near neutral pH. The compositions may not have any surfactants for maximum stability. The surfactants should be stable to hypohalous acid or hypohalous acid salt if long term storage is desired. If the solutions of the composition are generated prior to use, then surfactants having less stability may be used. Examples of surfactants having relatively good stability can be found in U.S. Pat. Nos. 6,413,925 and 5,851,421.

The composition may contain one or more surfactants selected from anionic, nonionic, cationic, ampholytic, amphoteric and zwitterionic surfactants and mixtures thereof. A typical listing of anionic, nonionic, ampholytic, and zwitterionic classes, and species of these surfactants, is given in U.S. Pat. No. 3,929,678 to Laughlin and Heuring. A list of suitable cationic surfactants is given in U.S. Pat. No. 4,259,217 to Murphy. Where present, ampholytic, amphoteric and zwitteronic surfactants are generally used in combination with one or more anionic and/or nonionic surfactants. The surfactants may be present at a level of from about 0% to 90%, or from about 0.001% to 50%, or from about 0.01% to 25% by weight.

Solvent

The composition of the invention may contain solvents. The solvents should be stable to hypohalous acid or hypohalous acid salt if long term storage is desired. However, even hypochlorite stable solvents are generally not stable in dilute hypochlorite compositions, such as those containing 40 to 200 ppm at near neutral pH. The compositions may not have any solvents, aside from water, for maximum stability. Suitable solvents might be hydrocarbons or esters not having any alcohol or olefinic groups. If the solutions of the composition are generated prior to use, then solvents having less stability may be used. The solvents can be present at a level of from 0.001% to 10%, or from 0.01% to 10%, or from 1% to 4% by weight.

Additional Adjuncts

The compositions optionally contain one or more of the following adjuncts: stain and soil repellants, lubricants, odor control agents, perfumes, fragrances and fragrance release agents, brighteners, and fluorescent whitening agents. Other adjuncts include, but are not limited to, acids, electrolytes, dyes and/or colorants, solubilizing materials, stabilizers, thickeners, defoamers, hydrotropes, cloud point modifiers, preservatives, and other polymers. For maximum stability, the compositions can contain no carboxylic acids, no carboxylic acids with hydroxyl or olefinic groups, no alcohols, no amines such as primary or secondary amines, no fragrances, no colorants, no flavorants, no preservatives, no odor or taste masking agents, and low salt content, for example less than 0.3 g/L, or less than 0.2 g/L.

The solubilizing materials, when used, include, but are not limited to, hydrotropes (e.g. water soluble salts of low molecular weight organic acids such as the sodium and/or potassium salts of toluene, cumene, and xylene sulfonic acid). The acids, when used, include, but are not limited to, mineral acids, and the like. Electrolytes, when used, include, calcium, sodium and potassium chloride. Preservatives, when used, include, but are not limited to, mildewstat or bacteriostat, methyl, ethyl and propyl parabens, phosphates such as trisodium phosphate, short chain organic acids (e.g. acetic, lactic and/or glycolic acids), bisguanidine compounds (e.g. Dantagard and/or Glydant) and/or short chain alcohols (e.g. ethanol and/or IPA). The mildewstat or bacteriostat includes, but is not limited to, mildewstats (including non-isothiazolone compounds) including Kathon GC.

Antimicrobial Agent

The composition of the invention may contain antimicrobial agents. The antimicrobial agents should be stable to hypohalous acid or hypohalous acid salt if long term storage is desired. If the solutions of the composition are generated prior to use, then antimicrobial agents having less stability may be used.

Builder/Buffer

The composition of the invention may contain a builder or buffer. The builder or buffer should be stable to hypohalous acid or hypohalous acid salt if long term storage is desired. If the solutions of the composition are generated prior to use, then builders or buffers having less stability may be used.

The composition may include a builder or buffer, which can be used as a pH adjusting agent or as a sequestering agent in the composition. The builder, buffer, or pH adjusting agent may be an inorganic buffer. Examples of buffers or pH adjusting agents include a hydroxide of alkali metal, a hydroxide of alkaline earth metal, an inorganic acid or a salt thereof, sodium hydroxide, potassium hydroxide, calcium hydroxide, hydrochloric acid, sulfuric acid, sodium sulfate, sodium nitrate, sodium chloride, sodium carbonate, potassium hydrogen carbonate, sodium hydrogen carbonate, magnesium sulfate, magnesium nitrate, magnesium chloride, magnesium carbonate, sodium triphosphate, potassium triphosphate, disodium hydrogenphosphate, dipotassium hydrogenphosphate, sodium dihydrogenphosphate, potassium dihydrogenphosphate, and sodium polyphosphate.

When employed, the builder, buffer, or pH adjusting agent comprises at least about 0.001% and typically about 0.001-0.5% of the composition. Preferably, the builder or buffer content is about 0.001-0.2%.

Water and pH

The water may be deionized, filtered to remove impurities including metals and organic carbon, purified by reverse osmosis, purified by distillation, or any combination thereof. Purified water may be prepared by a process selected from the group consisting of sodium cation exchange, hydrogen cation exchange, reverse osmosis, activated carbon treatment, UV light treatment, UVC, ozone treatment, chlorination, ultrafiltration, nanofiltration, electrodialysis, and a combination thereof. During preparation there may be a need for hygiene and segregation to prevent the introduction of compounds that are oxidized by hypochlorite since these become more important at low concentrations where the loss of a few ppm may be significant.

The composition may be adjusted for pH using a pH adjusting agent. Suitable pH adjusting agents include carbon dioxide, alkali metal carbonate, alkali metal bicarbonate, alkali metal silicates, alkali metal hydroxide, alkali phosphate salt, alkaline earth phosphate salt, alkali borate salt, hydrochloric acid, nitric acid, sulfuric acid, alkali metal hydrogen sulfate, organic sulfonic acids, sulfamic acid. The composition may have a pH from 1 to 13. The composition may have a pH from 2 to 12. The composition may have a pH from 2 to 5. The composition may have a pH from 5 to less than 8. The composition may have a pH between 4 and less than 8. The composition may have a pH between 6 and less than 8. The composition may have a pH greater than 5 and less than 9. The composition may have a pH greater than 5 and less than 8. The composition may have a pH from 6 to 7.5. The composition may have a pH from 9 to 13. The composition may have a pH from 10 to 12.

Method of Use

The composition may be dispersed into the air. The composition may be dispersed using an atomizer, a vaporizer, a nebulizer, a hose with laser created slits, or a spray device. The composition may be delivered on a continuous basis, such as with a humidifier. The composition may be delivered on a pulsed basis, such as with a canister on a timer. One spray device is an electrostatic sprayer, as described in WO0120988. The composition may be applied to skin surfaces. The composition may be delivered from a variety of containers, such as a dual chambered bottle, a trigger spray bottle, an aerosol canister, and a bleach pen.

The composition may be stored or shipped in a variety of containers, including glass, ABS, polycarbonate, high density polyethylene, low density polyethylene, high density polypropylene, low density polypropylene, polyethylene terephthalate, or polyvinylchloride. A variety of additives may affect the stability of the composition. For instance, the density of the polyethylene resin may be modified by co-polymerizing with a small amount of a short chain alkylene, e.g., butene, hexene or octene. Various other additives can be added, such as colorants, UV blockers, opacifying agents, and antioxidants, such as hindered phenols, e.g., BHT, Irganox 1010 (Ciba-Geigy A.G.), Irganox 1076 (Ciba-Geigy A.G.), Ionol (Shell Chemical Co.). Mold release agents and plasticizers can be added, especially to other types of plastics. The containers may have barrier films to increase storage stability. Suitable barrier films may include nylons, polyethylene terephthalate, fluorinated polyethylenes, and Barex (a copolymer of acrylonitrile and methylmethacrylate that is available from British Petroleum).

The composition may be applied to soft surfaces including clothing, bedding, upholstery, curtains, and carpets. The composition may be applied to soft surfaces by spraying, by wiping, by direct application, by immersion, or as part of the laundry washing process.

The composition may be applied to hard surfaces including kitchen surfaces, bathroom surfaces, walls, floors, outdoor surfaces, automobiles, countertops, food contact surfaces, toys, food products including fruits and vegetables. The composition may be applied to hard surfaces by spraying, by wiping, by direct application, by immersion, or as part of the normal cleaning process.

The composition may be applied on human and animal surfaces, including external skin areas and internal cavities. The composition may have lower skin sensitivity and may be appropriate to be taken orally or by inhalation. The composition may be applied to human and animal surfaces by spraying, by wiping, by direct application, by immersion, or as part of the normal treatment process. The composition may be applied as a thickened gel. The composition may be applied using a device to direct its application, such as a bleach pen. The composition may be applied as a wound dressing.

The composition may be applied with a nonwoven substrate, wipe or cleaning pad on inanimate, household surfaces, including floors, counter tops, furniture, windows, walls, and automobiles. The composition may be applied to baby and children's items, including toys, bottles, pacifiers, etc. The composition may be applied with a nonwoven substrate, brush, sponge, wipe or cleaning pad on human and animal surfaces, including external skin areas and internal cavities. Other surfaces include stainless steel, chrome, and shower enclosures. The nonwoven substrate, wipe or cleaning pad can be packaged individually or together in canisters, tubs, etc. The nonwoven substrate, wipe or cleaning pad can be used with the hand, or as part of a cleaning implement attached to a tool or motorized tool, such as one having a handle. Examples of tools using a nonwoven substrate, wipe or pad include U.S. Pat. No. 6,611,986 to Seals, WO00/71012 to Belt et al., U.S. Pat. App. 2002/0129835 to Pieroni and Foley, and WO00/27271 to Policicchio et al.

For certain uses, for example, for human and animal surfaces, the composition may be thickened. The composition may be thickened using surfactant thickening, polymer thickening for example clays, or other means. Thickening may allow more controlled application or application from a device. The composition may be thickened to a viscosity of from 40 to 10,000 cps. Examples of thickened and unthickened compositions can be found in U.S. Pat. No. 6,162,371, U.S. Pat. No. 6,066,614, U.S. Pat. No. 6,153,120, U.S. Pat. No. 6,037,318, U.S. Pat. No. 6,313,082, U.S. Pat. No. 5,688,435, U.S. Pat. No. 6,413,925, U.S. Pat. No. 6,297,209, U.S. Pat. No. 6,100,228, U.S. Pat. No. 5,916,859, U.S. Pat. No. 5,851,421, U.S. Pat. No. 5,688,756, U.S. Pat. No. 5,767,055, U.S. Pat. No. 5,055,219, and U.S. Pat. No. 5,075,029.

The anodic oxidation of chloride in an electrolysis cell results in the production of a number of oxychlorine ions including hypochlorite, chlorite, chlorate, and perchlorate. Chlorite is readily oxidized to chlorate. Perchlorate may be an undesirable contaminant in the environment due to its low reactivity, high mobility, and inhibition of thyroid function. The production of hypochlorite via chlorination of caustic water is not believed to result in the initial formation of perchlorate. This route may be advantageous for certain uses where minor amounts of perchlorate would be undesirable.

The composition may be prepared by mixing a solid composition with water. The solid composition may be a tablet, granular composition, paste, or other solid composition. The composition may be prepared by diluting a liquid composition with water. The water may be purified. The composition may be prepared by mixing two liquids, for example, from a dual chambered container or a dual chambered spray bottle.

The compositions of the invention can be diluted prior to use with tap water or water of higher purity. Preparation of dilute compositions for storage, for example as pre-diluted in bottles, may require water of higher purity. This higher purity water can be obtained by a variety of processes, including for example, distillation, filtering, sodium cation exchange (soft water), hydrogen cation exchange (deionized water without anion exchange), reverse osmosis, activated carbon treatment, ultrafiltration, nanofiltration, electrodialysis, and UV light treatment.

The compositions of the invention can be diluted prior to use from a concentrated liquid or solid composition. For instance, liquid sodium hypochlorite optionally containing surfactants or other additives of 5.25% available chlorine concentration (or above 0.5% concentration) can be diluted to below 500 ppm available chlorine concentration (or below 200 ppm). Tablets or powders having solid hypochlorite or hypochlorite generators can be dissolved in water to deliver compositions below 500 ppm concentration. Examples of compositions that can be diluted are described in U.S. Pat. No. 6,297,209, U.S. Pat. No. 6,100,228, U.S. Pat. No. 5,851,421, U.S. Pat. No. 5,688,756, U.S. Pat. No. 5,376,297, U.S. Pat. No. 5,034,150, U.S. Pat. No. 6,534,465, U.S. Pat. No. 6,503,877, U.S. Pat. No. 6,416,687, U.S. Pat. No. 6,180,583, and U.S. Pat. No. 6,051,676.

The compositions of the invention can be delivered as part of a multi-compartment delivery system, for example as described in U.S. Pat. No. 5,954,213, U.S. Pat. No. 5,316,159, WO2004/014760, U.S. Pat. No. 6,610,254, and U.S. Pat. No. 6,550,694.

The compositions of the invention can be used to purify water and make the water safe for consumption. The compositions of the invention can be used for a food rinse, for cleaning food-contact surfaces, and for toxicologically safe cleaning. This may involve the use of food-safe ingredients, GRAS ingredients, or ingredients with low toxicologically impact. Methods describing this use and possible compositions can be found in U.S. Pat. No. 6,455,086, U.S. Pat. No. 6,313,049, U.S. 2002/0132742, U.S. 2001/0014655, WO99/00025, and U.S. 2002/0151452.

The compositions of the invention can be used to sterilize medical instruments. Dilute hypochlorite will discolor or degrade tubing and other sensitive parts to less extent than concentrated hypochlorite. The compositions may be used in kidney dialysis machines or as an irrigating agent in endodontic treatment. The compositions of the invention can be used to kill tumor cells, affect tumor cell recognition and to induce apoptosis.

The compositions of the invention can be used in agricultural applications, for example, seed and seedling treatments, dormant sprays for fruit trees, stored grain treatments, dips or sprays for any post-harvest plant material and their containers, treatments for soil, either on the land or in containers, treatments for transportation and storage to market, treatments for transportation, storage, and display at market (retail or wholesale), treatments for import and export regulations, and treatments for preventing the accidental introduction of alien pest organisms. The compositions of the invention can be used for the meat, poultry, dairy, seafood, and aquaculture industries, for example, equipment treatments, living quarters treatments, dips or sprays for eggs and containers, dips or sprays for meat and containers, treatments for rendering operations, treatments for transportation and storage to market, treatments for transportation, storage, and display at market (retail or wholesale), treatments for import and export regulations, treatments for preventing alien pest organisms from crossing borders, treating disease on live animals (terrestrial or aquatic), including udder treatments, and dips or sprays for milking equipment, transfer lines, and containers. The compositions of the invention can be used for homeland security, for example, treatments for preventing the intentional introduction of alien pest organisms or deadly human or animal organisms.

The compositions of the invention can be used to preserve and maintain the freshness of freshly cut flowers and other cut plants. The compositions of the invention can be used to prevent the build-up of microorganisms that contribute to the decaying of stems and abscission and scenesing of leaves and flowers. The compositions of the invention can be used to preserve and extend the shelf life of freshly cut fruits and vegetables such as cut melon, cantaloupe, strawberry, potatoes, etc. The compositions of the invention can be used to eradicate hepatitis virus A from fresh strawberries and other fruits and vegetables. The compositions of the invention can be used in the sprout industry to treat seeds of various plants including alfalfa, wheat, barely and all other edible plants to control the spread of food-borne diseases such as Salmonella, E. coli, Campylobacter, etc. The compositions of the invention can be used in washing and treating shoes that have been moldy. The compositions of the invention can be used with sponges, cheese-cloth, paper towel and other non-woven articles to clean and remove and kill mold, bacteria and viruses from soft and hard surfaces. The compositions of the invention can be used to control mold in school. The compositions of the invention can be used as a spray or wipe product. The compositions of the invention can be used to control the spread of germs on hard surfaces in school. The compositions of the invention can be used to control the spread of hepatitis among jails. The compositions of the invention can be used in laundry to kill germs. The compositions of the invention can be used in long-term care centers and public gyms, where, for example, they can be applied as a spray or wipe product on hard surfaces to kill all germs that are transmitted via environmental surfaces and human. The compositions of the invention can be used in laundry to disinfect towels, and other articles that carry germs. The compositions of the invention can be used in public areas where, for example, they can be sprayed on a large scale in parks, streets, public places to control disease-causing agents such as SARS, calicivirus, enterovirus, FMD, and other viruses. The compositions of the invention can be used as wipes or spray to disinfect all environmental surfaces. The compositions of the invention can be used on ships and cruise ships where, for example, they can be used to control the spread of norwalk virus, calicivirus, and influenza virus. The compositions of the invention can be used to control cross contamination due to Salmonella and Campylobacter. The compositions of the invention can be used to protect from biological warfare where, for example, they can be used to spray on humans, (i.e., army personnel, medics, etc.) in case of potential presence of biological warfare agents such as Anthrax, BT, Sarin, Small Pox, and SARS, etc. The compositions of the invention can be used for disinfecting military vehicles, airplanes, and others. The compositions of the invention can be used to control the outbreak of infectious agents where, for example, they can be used to disinfect airplanes (inside and outside), trains, buses and all sort of transportation means to control the spread of pathogens. The compositions of the invention can be used to disinfect shoes (via a wipe or dipping or spraying) at airports and other ports of entry. The compositions of the invention can be used to control insects where, for example, they can be used as a spray to kill New Zealand Slug and other slugs or insects. The compositions of the invention can be used to kill fleas. The compositions of the invention can be used to control animal and insect pathogens where, for example, they can be used to control animal and bird viruses on hard surfaces and soft surfaces. Such viruses include SARS, bird flu virus, calicivirus, mad cow disease virus, parvovirus, feline viruses, etc. Also, they can be used to dip teats in to control various pathogens.

The composition may be part of an article of manufacture comprising: a container enclosing a liquid composition; a set of instructions; and a liquid composition comprising an allergen neutralizing agent selected from a group consisting of a hypohalous acid, a hypohalous acid salt, and a combination thereof; wherein said set of instructions comprises instructions to contact targets selected from a group consisting of hard surfaces, soft surfaces, or air with said liquid composition in its neat or diluted form to prevent allergic response, to prevent illness, or a combination thereof.

The composition may be part of an article of manufacture wherein said article of manufacture in addition to the usage instructions bears an additional indication comprising a term selected from the group consisting of: healthy, healthier, reduce the occurrence of illness, control the spread of illness in the home, protect your family from illness, keep your home healthier, keep your family well, break the cycle of illness in the home, reduce the risk of common illnesses, and combinations thereof.

The composition may be part of an article of manufacture, wherein said article of manufacture in addition to the usage instructions bears an additional indication comprising a term selected from the group consisting of: neutralizes mold allergens, denatures toxins from mold, neutralizes toxins from mold, neutralizes protein allergens, controls allergens, removes allergens by cleaning, removes allergens by wiping, removes allergens in the laundry, reduces respiratory illness, reduces hay fever, reduces absenteeism, denatures mold allergens, prevents allergenic reactions, prevents allergenic reaction in humans, prevents allergenic symptoms due to mold, kills mold, destroys mold spores, destroys mold spores that cause adverse health effects, proven to prevent mold-triggered allergic sensitization in humans, proven to prevent mold-triggered allergic sensitization in animals, reduces the risk of mold-triggered allergic sensitization, reduces the risk of mold-triggered allergic response, destroys mold spores that induce allergic symptoms, neutralizes mold specific antigens, and prevents non-immune inflammatory reactions to mold.

The composition may be part of an article of manufacture. The article of manufacture may include a set of instructions. The set of instructions may be used with a method of instructing the public by providing to the public a set of instructions for the use of an article of manufacture comprising a container and a liquid composition comprising an allergen neutralizing agent selected from a group consisting of a hypohalous acid, a hypohalous acid salt, and a combination thereof; wherein said set of instructions comprises instructions to contact targets selected from a group consisting of hard surfaces, soft surfaces, or air with said liquid composition in its neat or diluted form to prevent allergic response, to prevent illness, or a combination thereof. The instructions may relate to preventing the spread of illness with a liquid composition comprising a hypohalous acid salt composition. The method of instructing the public may include information that an allergic response represents a response to pollen, dust mite, or mold allergens. The set of instructions may be provided to the public via electronic and/or print media. The set of instructions may be posted at the point of sale adjacent the package. The set of instructions may be posted on a global computer network at an address associated with products from a group consisting of said liquid composition, said target surface, or a combination thereof.

The method of promoting the use of the liquid composition comprising an allergen neutralizing agent selected from a group consisting of a hypohalous acid, a hypohalous acid salt, and a combination thereof may include use instructions to prevent allergic response and/or illness, the method comprising the step of informing the public that the treatment of targets selected from a group consisting of hard surfaces, soft surfaces, or air with said composition reduces and/or prevents allergic response and/or illness. The method of promoting the use of the composition may include the step of informing the consumer via electronic and/or print media.

The use of the composition may include an in vivo test method for testing allergic response in animals, wherein said test method comprises the subcutaneous injection of allergens treated with a composition selected from a group consisting of a hypohalous acid, a hypohalous acid salt, and a combination thereof.

### EXAMPLES

Table I shows that diluted hypochlorite solutions have good stability at near neutral pH, especially when diluted with water relatively free from metal ions, salts, and total organic carbon (TOC) (Table II). The initial concentration of the concentrated sodium hypochlorite was 6.24% sodium hypochlorite and the stability samples were stored in 174 oz. Clorox® bleach bottles.

TABLE I

| | | Loss at | Loss at | Loss at |
|---|---|---|---|---|
| | Initial | 120° F. | 120° F. | 120° F. |
| | available | after | after | after |
| | chlorine | 9.9 days | 19.7 days | 30.7 days |
| Conc. | 84.2 (pH 7.53) | 17.1% | 23.7% | 33.4% (pH 7.84) |
| Hypochlorite | | | | |
| and tap water | | | | |
| Conc. | 83.0 (pH 7.53) | 19.3% | 22.8% | 26.0% (pH 8.11) |
| Hypochlorite | | | | |
| and Soft water^{a} | | | | |
| Conc. | 82.3 (pH 7.53) | 11.7% | 17.7% | 23.1% (pH 7.10) |
| Hypochlorite | | | | |
| and DDI water^{b} | | | | |
| Conc. | 83.3 (pH 7.53) | 10.9% | 16.9% | 22.0% (pH 7.18) |
| Hypochlorite | | | | |
| and DI water^{c} | | | | |
| Conc. | 83.0 (pH 7.53) | 11.0% | 15.9% | 19.4% (pH 7.52) |
| Hypochlorite | | | | |
| and RO water^{d} | | | | |
| Conc. | 85.0 (pH 7.53) | 11.8% | 17.3% | 22.1% (pH 7.20) |
| Hypochlorite | | | | |
| and RO/DI | | | | |
| water^{e} | | | | |

^{a}Soft water from a sodium cation exchange process.
^{b}DDI is deionized and then distilled water.
^{c}DI is from a hydrogen cation exchange process.
^{d}RO is from a reverse osmosis process.
^{e}RO/DI is from reverse osmosis and then a hydrogen cation exchange process.

TABLE II

| Water source | TOC, ppm | Cu, ppb |
|---|---|---|
| Tap Water | 0.702 | 129 |
| Soft Water | 3.030 | <70 |
| DDI Water | Not measured | Not measured |
| DI Water | 0.065 | <70 |
| RO Water | 0.052 | <70 |
| RO/DI Water | 0.059 | <70 |

Table III shows that diluted hypochlorite solutions have good stability at near neutral pH, especially when diluted with water relatively free from metal ions and salts. The solutions also have good stability in the presence of chelants, such as pyrophosphate and orthophosphate. The initial concentration of the concentrated sodium hypochlorite was 6.448% sodium hypochlorite.

TABLE III

| | Initial av. | | |
|---|---|---|---|
| | chlorine | Loss at 70° F. | Loss at 120° F. |
| | in ppm (pH) | after 27 days | after 27 days |
| Conc. Hypochlorite and | 79 (pH 7.6) | 7% | 52% |
| tap water | | | |
| Conc. Hypochlorite and | 77 (pH 7.5) | 0% | 22% |
| distilled deionized water | | | |
| Conc. Hypochlorite, | 81 (pH 7.6) | 6% | 25% |
| 23 ppm | | | |
| Orthophosphate, | | | |
| distilled deionized water | | | |
| Conc. Hypochlorite, | 80 (pH 7.6) | 4% | 29% |
| 11.5 ppm | | | |
| Pyrophosphate, | | | |
| distilled deionized water | | | |

Table IV shows compositions of the invention with impurity concentrations. Low concentrations of these impurities can enhance the stability of the compositions. In some cases, the initial concentrations of the impurities may be higher and the impurities may be made less reactive or inert over time. In these cases, the compositions may have increased stability upon aging.

TABLE IV

| | |
|---|---|
| Diluted | Diluted |
| hypochlorite | hypochlorite |

| Available chlorine, ppm | 200 | ppm | 40 | ppm |
|---|---|---|---|---|
| pH | 7.9 | | 5.1 | |
| Copper | <100 | ppb | <80 | ppb |
| Nickel | <10 | ppb | <8 | ppb |
| Cobalt | <30 | ppb | <20 | ppb |
| Total organic carbon | <500 | ppb | <200 | ppb |

Table V shows that dust mite allergens are effectively denatured with diluted hypochlorite solutions down to 5 ppm available chlorine. The pH obtained for diluted hypochlorite solution at 4 ppm was 6.51. The compositions are also effective against allergens within 30 seconds.

Product efficacy screening was performed by using a modified antibody capture ELISA (where a recombinant antigen is coated onto polystyrene, the product is added directly to predetermined wells and incubated for a selected period of time, the results of the product treated wells are compared against those of untreated wells, the concentration is calculated against a standard curve). This method differs from the antigen capture ELISA in that product interference which affected antibody-antigen complex is not considered because the product is added directly to the antigen/allergen, the wells are washed of excess product and the labeled antibody is incubated onto the remains of the antigen. Protein fragmentation was revealed by SDS-PAGE method and loss of Allergenic activity (antibody binding to antigen) was observed in Western blot (immunoblot).

TABLE V

| | Available chlorine, | Dust mite allergen, % |
|---|---|---|
| | ppm | reduction |
| Diluted hypochlorite | 0.77 | 75 |
| | 4.0 | 98 |
| | 7.8 | 99 |
| | 19.4 | 100 |
| | 38.4 | 100 |
| | 57.7 | 100 |
| | 77 | 100 (30 sec) |

Table VI shows that diluted hypochlorite is effective at sanitizing and disinfecting as measured by efficacy against Staphylococcus aureus. Tests were conducted using the AOAC Germicidal Spray Products test method (AOAC 961.02, 15th edition, SOP No. 001-057-06). An approximate 48-hour suspension of Staphylococcus aureus grown up in AOAC Synthetic Broth was used for testing. The culture concentration was adjusted to yield a target of 4×10⁴ per slide once dried. For the runs to be conducted with organic soil load, a separate bacteria suspension was prepared with fetal bovine serum where the serum load was 5%. A volume of 0.01 ml was inoculated per glass slide. A sterile bent needle was used to spread the inoculum to within ⅛″ from the edge. For each inoculation run, the slides were dried in the 35° C. incubator until completely dry. Prior to testing, bottle caps were replaced with trigger sprayers. The triggers were primed and testing was started by spraying the contaminated surfaces from 6-8 cm distance for 2-3 seconds. The surface was completely wet by about 3-4 full pumps. The amount of product that was dispensed per trigger ranged from 2.24 g to 2.90 g. For the samples that were pipetted onto the contaminated surfaces, the dispensing volume was between 2.5 ml per slide (with filter paper) and 5 ml per slide (without filter paper).

TABLE VI

| | | | Sample |
|---|---|---|---|
| | | | with |
| | Available chlorine | | residual |
| | in ppm | pH | bacteria |
| Diluted | 707.6 | 9.70 | 0/60 |
| hypochlorite | | | |
| Diluted | 63.4 | 7.36 | 0/60 |
| hypochlorite | | | |
| After storage | | | |
| 120 F. for 1 | | | |
| month | | | |

Table VII shows that the compositions are effective at killing a variety of viruses and spores.

TABLE VII

| | Diluted hypochlorite |
|---|---|
| Polio I Virus | Effective |
| Influenza A Virus | Effective |

The compositions are effective at controlling mold growth. Diluted hypochlorite tested against penicillium mold in a petri dish gave growth inhibition.

The dilute hypochlorite compositions are effective at controlling odors. Dilute hypochlorite can control odors by both killing the odor-causing bacteria as well as by oxidizing the odor molecules themselves, breaking them down into smaller, odorless components. An initial test was done using garlic juice in small plastic containers. A drop of garlic juice was placed in each of two plastic containers at room temperature and allowed to equilibrate for 10 minutes. The containers are then opened and one is sprayed with dilute hypochlorite and one with plain water. The containers were then closed and again allowed to equilibrate for 10 minutes. Then a corner of the container is opened to smell the contents. The containers sprayed with dilute hypochlorite had less garlic odor than the one sprayed with water.

The compositions of the invention can give minimal fabric damage compared to other hypochlorite compositions. Cotton, rayon, and wool were sprayed with dilute hypochlorite until damp and allowed to dry between sprayings. Test was repeated for upwards of 20+ sprays. No visible damage was observed. Swatches of bleach sensitive blue-dyed cotton (Intralite Turquoise GL) were soaked in dilute hypochlorite solutions. Swatches showed no discoloration for several hours. Some bleaching was observed when soaked for longer times and was easily observable after 24 hours.

The composition of the invention was found to kill Aspergillus fumigatus Conidia spores in solution and to inactivate Aspergillus fumigatus Conidia antigen in solution. The composition was also tested on hard surfaces. The composition of the invention was found to reduce mold growth on drywall 6 logs compared to water (none). The composition of the invention was found to reduce mold growth on plywood 6 logs compared to water (none). The composition of the invention was found to reduce mold growth on oriented strand board more than 6 logs compared to water (none). The compositions of the invention were tested for in vivo allergic response in humans, wherein said test method comprises the subcutaneous injection of allergens treated with the composition. The residue after treatment on oriented strand board was evaluated by prick skin testing on test subjects who had a history of positive skin prick to Aspergillus fumigatus.

Results from the in vivo testing suggest that the inventive compositions will reduce or prevent respiratory ailments caused by allergens and reduce or prevent allergies.

The stability results for dilute hypochlorite solutions diluted with deionized distilled water and adjusted to pH 7 are given below in Table VIII for several buffering systems and concentrations of approximately 40 ppm, 75 ppm, and 150 ppm sodium hypochlorite. Citric acid, an organic hydroxyl containing acid has poor stability with or without sodium dihydrogen phosphate. However, hypochlorite buffered with hydrochloric acid or 3,3-dimethylglutaric acid, which has no enolizable hydrogens has good stability.

Besides metal contaminants, the compositions may also be substantially free of certain organic contaminants, such as surfactants or alcohols or amino compounds, or thiol compounds, or hydroxyacids, or olefinic compounds or fragrances. In some cases the composition may be substantially free of organic acids with enolizable hydrogens. The compositions may also have a low concentration of inorganic salts of less than 0.3 g/L.

TABLE VIII

| | | | % Remaining | | | |
|---|---|---|---|---|---|---|
| Storage at 120° F. | Initial | | 7 days | 14 days | 21 days | 28 days |
| NaOCl diluted from 3.9% with | 42.3 | ppm | 20% | 3% | 1% | 1% |
| deionized distilled water and | | | | | | |
| 0.1M Citric Acid to pH 7.01 | | | | | | |
| NaOCl diluted from 3.9% with | 77.5 | ppm | 3% | 1% | 1% | 1% |
| deionized distilled water and | | | | | | |
| 0.1M Citric Acid to pH 7.01 | | | | | | |
| NaOCl diluted from 3.9% with | 148.1 | ppm | 1% | 0% | 0% | 0% |
| deionized distilled water and | | | | | | |
| 0.1M Citric Acid to pH 7.02 | | | | | | |
| NaOCl diluted from 3.9% with | 41.5 | ppm | 26% | 11% | 3% | 1% |
| deionized distilled water and | | | | | | |
| 0.1M NaH₂PO₄ and 0.1M | | | | | | |
| Citric Acid to pH 7.03 | | | | | | |
| NaOCl diluted from 3.9% with | 78.7 | ppm | 12% | 1% | 1% | 1% |
| deionized distilled water and | | | | | | |
| 0.1M NaH₂PO₄ and 0.1M | | | | | | |
| Citric Acid to pH 7. 01 | | | | | | |
| NaOCl diluted from 3.9% with | 147.9 | ppm | 1% | 0% | 0% | 0% |
| deionized distilled water and | | | | | | |
| 0.1M NaH₂PO₄ and 0.1M | | | | | | |
| Citric Acid to pH 7.03 | | | | | | |
| NaOCl diluted from 3.9% with | 42.5 | ppm | 96% | 88% | 87% | 86% |
| deionized distilled water and | | | | | | |
| 0.1M HCl to pH 7.03 | | | | | | |
| NaOCl diluted from 3.9% with | 78.1 | ppm | 97% | 91% | 90% | 87% |
| deionized distilled water and | | | | | | |
| 0.1M HCl to pH 7.02 | | | | | | |
| NaOCl diluted from 3.9% with | 145.8 | ppm | 93% | 85% | 82% | 80% |
| deionized distilled water and | | | | | | |
| 0.1M HCl to pH 7.02 | | | | | | |
| NaOCl diluted from 3.9% with | 42.6 | ppm | | 87% | 83% | 82% |
| deionized distilled water and | | | | | | |
| 0.1M dimethylglutaric acid to | | | | | | |
| pH 7.02 | | | | | | |
| NaOCl diluted from 3.9% with | 77.9 | ppm. | | 90% | 84% | 80% |
| deionized distilled water and | | | | | | |
| 0.1M dimethylglutaric acid to | | | | | | |
| pH 7.03 | | | | | | |
| NaOCl diluted from 3.9% with | 149.5 | ppm | | 82% | 77% | 73% |
| deionized distilled water and | | | | | | |
| 0.1M dimethylglutaric acid to | | | | | | |
| pH 7.01 | | | | | | |

This invention has been described herein in considerable detail to provide those skilled in the art with information relevant to apply the novel principles and to construct and use such specialized components as are required. However, it is to be understood that the invention can be carried out by different equipment, materials and devices, and that various modifications, both as to the equipment and operating procedures, can be accomplished without departing from the scope of the invention itself. As such, these changes and modifications are properly, equitably, and intended to be, within the full range of equivalence of the following claims.

We claim:

1. A method for producing a stable dilute composition, said composition selected from the group consisting of hypohalous acid, hypohalous acid salt, and combinations thereof, said method comprising the steps of:
a. providing a source of active material from the group consisting of hypohalous acid, hypohalous acid salt, hypohalous acid generating species, hypohalous acid salt generating species, and combinations thereof; and
b. diluting said source with purified water and inorganic buffer to give said stable dilute composition having a pH between 4 and less than 8;
c. wherein said stable dilute composition has an available chlorine concentration of between 40 ppm to about 400 ppm;
d. wherein said stable dilute composition does not contain additives selected from the group consisting of surfactants, alcohols, amino compounds, thio compounds, hydroxyacids, olefinic compounds, fragrances or combinations thereof.

2. A method for producing a stable dilute composition, said composition selected from the group consisting of hypohalous acid, hypohalous acid salt, and combinations thereof, said method comprising the steps of:
a. providing a source of active material from the group consisting of hypohalous acid, hypohalous acid salt, hypohalous acid generating species, hypohalous acid salt generating species, and combinations thereof; and
b. diluting said source with purified water to give said stable dilute composition;
c. wherein said stable dilute composition has an available chlorine concentration of between 40 ppm to about 1200 ppm;
d. wherein said stable dilute composition does not contain additives selected from the group consisting of surfactants, alcohols, hydroxyacids, fragrances or combinations thereof.

3. A method for producing a stable dilute composition, said composition selected from the group consisting of hypohalous acid, hypohalous acid salt, and combinations thereof, said method comprising the steps of:
a. preparing a first solution having an active halogen content of greater than about 0.5% as available chlorine; and
b. diluting said first solution with purified water to give a second solution;
c. wherein said second solution has an available chlorine concentration of between 40 ppm to about 400 ppm;
d. wherein said second solution retains at least 50% of the available chlorine concentration at a storage temperature of 120° F. over 27 days;
e. wherein said stable dilute composition does not contain additives selected from the group consisting of surfactants, alcohols, hydroxyacids, fragrances or combinations thereof.

4. The method of claim 3, wherein said second solution retains at least 70% of the available chlorine concentration at a storage temperature of 120° F. over 27 days.

5. The method of claim 3, wherein said second solution has a pH greater than pH 5 and less than pH 9.

6. The method of claim 3, wherein said second solution has a pH greater than pH 5 and less than pH 8.

7. The method of claim 3, wherein said second solution has an available chlorine concentration of less than 200 ppm.

8. The method of claim 3, wherein said second solution has an available chlorine concentration of less than 100 ppm.

9. The method of claim 3, wherein said purified water is prepared by a process selected from the group consisting of sodium cation exchange, hydrogen cation exchange, reverse osmosis, activated carbon treatment, UV light treatment, ultrafiltration, nanofiltration, electrodialysis, and a combination thereof.

10. The method of claim 3, wherein said purified water is prepared by an ion-exchange process.

11. The method of claim 3, wherein said second solution is used to treat allergens.

12. The method of claim 3, wherein said second solution additionally comprises a pH adjusting agent.

13. The method of claim 12, wherein said pH adjusting agent is selected from the group consisting of carbon dioxide, alkali metal carbonate, alkali metal bicarbonate, alkali metal silicates, alkali metal hydroxide, alkali phosphate salt, alkaline earth phosphate salt, alkali borate salt, hydrochloric acid, nitric acid, sulfuric acid, alkali metal hydrogen sulfate, organic sulfonic acids, sulfamic acid, and mixtures thereof.

14. The method of claim 12, wherein said pH adjusting agent is selected from a carboxylic acid having no hydroxyl groups or olefinic groups.

15. The method of claim 3, wherein said second solution has a salt concentration of less than 0.3 g/L.
